SEQUENCE LISTING (18 sequences)

SEQ ID NO: 1 - DNA, 31 bp, Artificial Sequence
  Synthetic Oligonucleotide
ttagggctagcctcggaattgagaatgatgg

SEQ ID NO: 2 - DNA, 38 bp, Artificial Sequence
  Synthetic Oligonucleotide
guccuaagguagcgaaauuccuugucggguaaguuccg

SEQ ID NO: 3 - DNA, 42 bp, Artificial Sequence
  Synthetic Oligonucleotide
gacucaacacggggaaacucaccagguccagacacaauaagg

SEQ ID NO: 4 - DNA, 24 bp, Artificial Sequence
  Synthetic Oligonucleotide
uaaccguucucaucgcucuacgga

SEQ ID NO: 5 - DNA, 23 bp, Artificial Sequence
  Synthetic Oligonucleotide
ucccccgcuacccgguacguucc

SEQ ID NO: 6 - DNA, 15 bp, Artificial Sequence
  Synthetic Oligonucleotide
ctcaattccgaggct

SEQ ID NO: 7 - DNA, 16 bp, Artificial Sequence
  Synthetic Oligonucleotide
cgacaaggaauuucgc

SEQ ID NO: 8 - DNA, 22 bp, Artificial Sequence
  Synthetic Oligonucleotide
gtctggacctggtgagtttccc

SEQ ID NO: 9 - DNA, 14 bp, Artificial Sequence
  Synthetic Oligonucleotide
agagcgaugagaac

SEQ ID NO: 10 - DNA, 13 bp, Artificial Sequence
  Synthetic Oligonucleotide
guaccggguagcg

SEQ ID NO: 11 - DNA, 27 bp, Artificial Sequence
  Synthetic Oligonucleotide
tgcgtgctcaattccgaggctcacgca

SEQ ID NO: 12 - DNA, 28 bp, Artificial Sequence
  Synthetic Oligonucleotide
ttgcgtgctcaattccgaggctcacgca

SEQ ID NO: 13 - DNA, 32 bp, Artificial Sequence
  Synthetic Oligonucleotide
cccagcaccgacaaggaauuucgcgtgctggg

SEQ ID NO: 14 - DNA, 38 bp, Artificial Sequence
  Synthetic Oligonucleotide
ccgaggacgtctggacctggtgagtttcccgtcctcgg

SEQ ID NO: 15 - DNA, 24 bp, Artificial Sequence
  Synthetic Oligonucleotide
ccgacagagcgaugagaacgucgg

SEQ ID NO: 16 - DNA, 23 bp, Artificial Sequence
  Synthetic Oligonucleotide
ccgagguaccggguagcgcucgg

SEQ ID NO: 17 - DNA, 25 bp, Artificial Sequence
  Synthetic Oligonucleotide
uaaccguucucaucgcucuacggac

SEQ ID NO: 18 - DNA, 24 bp, Artificial Sequence
  Synthetic Oligonucleotide
ucccccgcuacccgguacguuccc

(19) United States
(12) Patent Application Publication
(10) Pub. No.: US 2013/0040859 A1
(43) Pub. Date: Feb. 14, 2013

(54) SPECTRALLY-RESOLVED CHEMILUMINESCENT PROBES FOR SENSITIVE MULTIPLEX MOLECULAR QUANTIFICATION

(71) Applicants: Kenneth A. BROWNE, Poway, CA (US); Ian WEEKS, Cardiff (GB); Richard C. BROWN, Cardiff (GB)

(72) Inventors: Kenneth A. BROWNE, Poway, CA (US); Ian WEEKS, Cardiff (GB); Richard C. BROWN, Cardiff (GB)

(73) Assignee: GEN-PROBE INCORPORATED, San Diego, CA (US)

(21) Appl. No.: 13/564,550

(22) Filed: Aug. 1, 2012

Related U.S. Application Data
(60) Provisional application No. 61/521,288, filed on Aug. 8, 2011.

(51) Int. Cl. C07H 21/04; C40B 40/06; G01N 21/76

(52) U.S. Cl. 506/16; 536/24.32; 435/6.11

(57) ABSTRACT

Hybrid luminescent probes emit light of distinct wavelength ranges and intensities upon energy transfer from an in-common, acridinium ester chemiluminophore to a coupled luminophore. The probes include: (1) a target binding region with a base sequence that is substantially complementary to a portion of the target nucleic acid sequence; (2) an acridinium ester (AE) moiety attached to a first region flanking the target binding region; (3) a luminophore coupled to the AE moiety to allow energy transfer from an acridone moiety, produced by a chemical triggering of the AE moiety, to the luminophore; and (4) a quencher moiety attached to a second region flanking the target binding region, such that the first and second flanking regions are on the opposite sides of the target binding region. The probes are particularly useful in homogeneous assays for sensitive, multiplex quantification of nucleic acid target sequences without prior enzymatic amplification.

### CROSS REFERENCE TO RELATED APPLICATIONS

This application claims the benefit under 35 U.S.C. §119(e) to provisional patent application Ser. No. 61/521,288, filed Aug. 8, 2011, which is hereby incorporated by reference.

### FIELD OF THE INVENTION

This invention generally relates to the field of molecular diagnostics. More specifically, the invention relates to the detection and quantification of nucleic acid targets in a sample using spectrally resolved chemiluminescent probes.

### BACKGROUND OF THE INVENTION

Hybridization of nucleic acid probes to complementary nucleic acid targets has been critical to the development of rapid molecular detection of specific sequences in fields ranging from clinical diagnostics to forensics and environmental monitoring. Sensitive probe labels evolved from radioisotopes to chemiluminophores and fluorophores, and discrimination of bound from unbound probes improved from heterogeneous formats, requiring substantial washing of hybridized sequences to remove unhybridized probe, to an increased reliance on homogeneous formats that do not require such steps (Spiegelman et al., Proc. Natl. Acad. Sci. U.S.A. 1971, 68:2843-45; Southern, E. M., J. Mol. Biol. 1975, 98:503-17; Tyagi et al., Nat. Biotechnol. 1996, 14:303-08; Nelson et al., Biochemistry 1996, 35:8429-38). Detection of high concentrations (e.g., high nanomolar) of multiple nucleic acid targets through implementation of molecular beacons (MBs) and related fluorescent probe technologies has increased the value of homogeneous assays in terms of information returned, cost and time, especially if preceded by amplification of the target sequence (Tyagi 1996, supra; Tyagi et al., Nat. Biotechnol. 2000, 18:1191-96). MBs function as homogeneous probes by their ability to change secondary structure and, thus, modulate signaling activity. Self-complementary nucleotide (nt) sequences near each terminus of MBs promote a stem-loop structure that constrains the fluorophore (F) at one terminus to the proximity of a quenching moiety at the opposite terminus, leading to low emission of light upon excitation of the fluorophore in the absence of target. Hybridization of a complementary target to the loop sequence forces the stem sequences as well as the fluorophore and quencher apart, thereby permitting signaling the presence of a specific target through increased detectable fluorescence.

Excitation light and nonspecific fluorescence in a sample contribute to background fluorescence while these sources of background are absent in chemiluminescent systems. Lower background signals from a chemically-initiated, rather than from a photonically-initiated, electronically-excited state molecule allow detection of emissions to be much more sensitive than by fluorescence. Acridinium ester (AE) probes, for example, exhibit the ability to homogeneously report low concentrations (down to high picomolar) of complementary target over a wide dynamic range in the presence of closely related targets (Nelson, supra). Light from chemiluminescent reactions has traditionally been collected over entire luminophore emission ranges (within the limits of the detector), reducing the number of co-occurring targets that can be simultaneously quantified as a function of wavelength.

Various methods have been developed to permit the simultaneous detection of more than one target using chemi- or bioluminescence. For example, in a multi-step, two protein analyte, heterogeneous assay, Adamczyk et al. stimulated an aequorin label with Ca²⁺, recorded the luminescence, then initiated and recorded luminescence from an acridinium-9-carboxamide label with alkaline peroxide (Adamczyk et al., Bioorg. Med. Chem. Lett. 2002, 12:395-98). Previously, we demonstrated that two analyte nucleic acids could be homogeneously and simultaneously quantified down to 0.5-5 fmol by using a pair of HICS probes with distinct AE labels (either AE or 2,7-dimethoxyAE) and independently detecting either low or high wavelength emissions, respectively (U.S. Pat. No. 7,169,554). However, considerable effort is involved in synthesis of unique compounds such as the 2,7-dimethoxyAE label, reducing throughput for design, synthesis and screening of large numbers of wavelength-distinguishable chemiluminescent probes.

Examples of energy transfer (ET) from chemiluminophores to fluorophores have been reported for analyte detection, mostly as intermolecular combinations. Patel and Campbell used chemiluminescence ET from aminobutylethyl-isoluminol-labeled haptens to fluorescein-labeled antibodies to quantify small molecules in serum or tissues (Patel, A. & Campbell, A. K., Clin. Chem. 1983, 29:1604-08). Heller and Morrison designed pairs of nucleic acid probes labeled with luminol or rhodamine at their termini such that only upon hybridization of both probes to a complementary sequence would the chemiluminophore and the fluorophore be proximal, capable of ET and reporting of the analyte (Heller, M. J. & Morrison, L. E., in RAPID DETECTION AND IDENTIFICATION OF INFECTIOUS AGENTS, Kingsbury, D. T. & Falkow, S., eds.; Academic Press: New York, 1985, pp 245-56). Schaap et al. reported enzymatic triggering of a dioxetane substrate resulting in ET to a fluorescein conjugate embedded in a cetyltrimethylammonium bromide micelle, leading to a 400-fold increase in detected light compared to that from the dioxetane alone (Schaap et al., Clin. Chem. 1989, 35:1863-64). In a heterogeneous assay, Zhang et al. used binary nucleic acid probes labeled with luminol and a fluorophore (but fluorescein instead of rhodamine) and a third unlabeled probe to bind to an aptamer sequence and signal the presence of a bound ATP molecule (Zhang et al., Anal. Chem. 2009, 81:8695-8701). Slightly different from the above examples, Soukka et al. disclosed a system in which electrochemiluminescence energy is transferred to a fluorophore acceptor and the emitted radiation detected (U.S. Pat. No. 7,790,392). Departing from the above works, and of the intramolecular nature of the present probes, the disclosure of Jiang et al. describes ET from AE molecules to fluorophores covalently coupled to the peripositions of the AE nucleus (U.S. Pat. No. 6,165,800). These AE-fluorophore constructs may have been inspired by the chemiluminescent ET work of White and Roswell with hydrazide-fluorophore conjugates (White, E. H. & Roswell, D. F., J. Am. Chem. Soc. 1967, 89:3944-45).

Despite the technological advances described above, there remains a need for new chemiluminescent probes with different emission profiles that could be generated rapidly and inexpensively without a complicated synthetic route. The instant invention discloses rapid generation of multiple chemiluminescent probes with different wavelength emissions by solid phase oligonucleotide synthesis and demonstrates utility of these wavelength-shifted (ws) probes in multiple analyte detection.

### SUMMARY OF THE INVENTION

In one aspect, the present invention provides a chemiluminescent probe for detecting a target nucleic acid sequence in a sample, e.g., a biological or environmental sample. The probe includes at least the following structural elements: (1) a target binding region with a base sequence that is substantially complementary to a portion of the target nucleic acid sequence; (2) an acridinium ester (AE) moiety attached to a first region flanking the target binding region; (3) a luminophore coupled to the AE moiety to allow energy transfer from an acridone moiety, produced by a chemical triggering of the AE moiety, to the luminophore; and (4) a quencher moiety attached to a second region flanking the target binding region, such that the first and second flanking regions are on the opposite sides of the target binding region. In the absence of the target nucleic acid sequence, the probe is in an inactive conformation, wherein the quencher moiety is sufficiently close to the AE-coupled luminophore to substantially block its emission. In the presence of the target nucleic acid sequence, the probe assumes an active conformation, wherein the quencher moiety moves sufficiently far from the AE-coupled luminophore so that emission from the luminophore can be detected following the chemical triggering of the AE moiety.

The oligonucleotide structure of the chemiluminescent probe is preferably akin to that of hybridization induced chemiluminescent signal (HICS) probe, a molecular beacon, a molecular torch, or a hybridization switch probe. In some embodiments, the AE moiety is conjugated to a site proximal to a terminus of a stem-loop oligonucleotide, and a quenching moiety is attached proximal to the opposite terminus of the oligonucleotide. Alternatively, the AE and quenching moieties may be conjugated to sites that are proximal to the stem-loop junction or at other positions on the stem of a stem-loop oligonucleotide. In some embodiments, the AE moiety is conjugated through an acridinium position other than C9 to a stem-loop oligonucleotide. For example, the AE moiety may be conjugated through the N10 acridinium position (N-linked) to a stem-loop oligonucleotide. Preferably, the AE moiety is a 9-(2,6-dibromophenoxycarbonyl)-10-(3-carbonylpropyl)acridinium salt (e.g., iodide). In some embodiments, the luminophore is a fluorescent dye, e.g., a fluorescein dye, a rhodamine dye, or a cyanine dye. The luminophore may be coupled to the AE moiety directly or via a linker. Preferably, the luminophore is attached to the oligonucleotide backbone at or near the first region flanking the target binding region, so that the AE moiety and the luminophore are linked to each other by a small oligonucleotide fragment of the probe. The quencher moiety may include a wide variety of molecules capable of blocking emission from the AE-coupled luminophore. Without limitation, examples of such molecules include Dabcyl and Black Hole Quencher 2 (BHQ-2).

As noted above, one of the objects of the present invention is to provide an improved method for detecting and quantifying multiple nucleic acid targets in the same sample. The target nucleic acid sequences can be from a mammalian (e.g., human) organism, a bacterium, a fungus, a virus, or any other organism of interest. For example, simultaneous quantitative determination of multiple infectious organisms represents one attractive application of the present invention. In some embodiments, the target nucleic acid sequences are selected from the group consisting of an Enterococcus target sequence, a pan-fungal target sequence, and a Neisseria gonorrhoeae (N. gonorrhoeae) target sequence.

The Enterococcus target nucleic acid sequence may include a fragment of Enterococcus faecalis (E. faecalis) 23S rRNA corresponding to nucleotides 866-896 and consisting of SEQ ID NO:1, allowing for a DNA equivalent thereof. In some embodiments, an Enterococcus probe may include a stem-loop oligonucleotide having a target complementary base sequence consisting of SEQ ID NO:6, allowing for an RNA equivalent thereof. In preferred embodiments, the stem-loop oligonucleotide may include a base sequence consisting of any one of SEQ ID NOs:11 and 12, allowing for RNA equivalents thereof.

The pan-fungal target nucleic acid sequence may include a fragment of Candida albicans (C. albicans) 18S rRNA corresponding to nucleotides 1174-1217 and consisting of SEQ ID NO:3, allowing for a DNA equivalent thereof. In some embodiments, a pan-fungal probe may include a stem-loop oligonucleotide having a target complementary sequence consisting of SEQ ID NO:8, allowing for an RNA equivalent thereof. In preferred embodiments, the stem-loop oligonucleotide may include a base sequence consisting of SEQ ID NO:14, allowing for an RNA equivalent thereof.

The N. gonorrhoeae target nucleic acid sequence may include a 16S rRNA fragment corresponding to nucleotides 128-150 and consisting of SEQ ID NO:5, allowing for a DNA equivalent thereof. In some embodiments, an N. gonorrhoeae probe may include a stem-loop oligonucleotide having a target complementary base sequence consisting of SEQ ID NO:10, allowing for a DNA equivalent thereof. In preferred embodiments, the stem-loop oligonucleotide may include a base sequence consisting of SEQ ID NO:16, allowing for a DNA equivalent thereof.

In some embodiments, the chemiluminescent probes of the present invention provide a detection range of up to about 3 log units, preferably from about 200 amol to about 200 fmol, from about 500 amol to about 200 fmol, from about 2 fmol to about 200 fmol, from about 500 amol to about 50 fmol, or from about 500 amol to about 5 fmol. Multiple wavelength-shifted probes are capable of detecting two, three, four, five, or more different target sequences, provided the probes' emission profiles are sufficiently distinct from each other to permit spectral and/or temporal resolution.

In another aspect, the present invention provides a kit for detecting and/or quantifying a target nucleic acid sequence in a sample that includes a wavelength-shifted chemiluminescent probe according to the present invention and reagent means for triggering a chemiluminescence reaction. In some embodiments, the kit may detect and/or quantify a plurality of target nucleic acid sequences in a sample. Such a kit preferably contains reagent means triggering a chemiluminescence reaction and a plurality of wavelength-shifted chemiluminescent probes having sufficiently different emission profiles to allow spectral and/or temporal resolution of their luminescence emissions. In some embodiments, the plurality of wavelength-shifted probes may include different luminophores coupled to the same AE moiety. Alternatively, different AE moieties may also be employed. In some embodiments, the AE moiety is conjugated through an acridinium position other than C9 to a stem-loop oligonucleotide. For example, the AE moiety may be conjugated through the N10 acridinium position (N-linked) to a stem-loop oligonucleotide. Preferably, the AE moiety is a 9-(2,6-dibromophenoxycarbonyl)-10-(3-carbonylpropyl)acridinium salt (e.g., iodide). In some embodiments, the luminophore is a fluorescent dye, e.g., a fluorescein dye, a rhodamine dye, or a cyanine dye. The quencher moiety may include a wide variety of molecules capable of blocking emission from the AE-coupled luminophore. Without limitation, examples of such molecules include Dabcyl and Black Hole Quencher 2 (BHQ-2).

### BRIEF DESCRIPTION OF THE DRAWINGS

FIG. 1 illustrates interactions and excitations/emissions of self-quenching chemiluminescent (A) and wavelength-shifted chemiluminescent (B) hairpin probes (referred to herein as HICS and wsHICS probes, respectively) with complementary target nucleic acids. Solid lines between nucleic acid strands represent base pairing; F and Q represent fluorophore and quencher moieties, respectively; and AE represents an acridinium ester chemiluminescent moiety. Hybridization of a HICS probe loop to a complementary target nucleic acid forces the stem arms and, hence, the quenching moiety (Q) and the luminophore (A) AE or (B) AE-fluorophore (AE-F) construct apart. Addition of oxidizing agent and increased alkalinity initiates light emission characteristic of (A) the electronically-excited species (acridone*) or (B) the F after ET from the acridone*. In the absence of target, the stem arms remain hybridized, Q and the luminophore remain proximal, and little energy as light emissions is detected.

FIG. 2 shows the chemical structures of (A) C-linked and (B) N-linked AE NHS ester label compounds.

FIG. 3 compares time-resolved spectrograms following chemical initiation of: (A) 50 pmol EfaB874-888(−) HICS1; (B) 100 pmol EfaB874-888(−) wsHICS10a (AE-fluorescein); (C) 50 pmol EfaB874-888(−) wsHICS11a (AE-tetramethylrhodamine); and (D) 200 pmol EfaB874-888(−) wsHICS12a (AE-Cy5) pre-hybridized to equimolar amounts of their complementary targets in hybridization reagent. The curves show the counts per second (cps) summed over the preceding 0.4 sec interval (A, B) or the preceding 5 sec interval (C, D). Insets show results for probes in the absence of target with the same axis scales.

FIG. 4 illustrates representative time courses of chemiluminescent emissions for: (A) 0.5 pmol EfaB874-888(−) HICS1; and (B) 0.5 pmol EfaB874-888(−) wsHICS11 a, each plus 1 pmol EfaB866-896(+) target sequence. Each point shows the cps summed over a 0.05 sec interval. Insets are output from 0.5 pmol probes in the absence of target. Lines through data points are non-linear best fits to a linear combination of exponential functions (Eq. 1). Low magnitude dotted lines are output from 0.5 pmol probes in the absence of target.

FIG. 5 demonstrates background subtracted emissions from 200 fmol of both EcoB1932-1947(−) HICS18 and CalA1185-1206(−) wsHICS88 probes versus synthetic EcoB1921-1958(+) (◯) and CalA1174-1217(+) (□) target concentrations in equal volumes of seawater and hybridization reagent. Open symbols (◯,□) are in the presence of indicated target, whereas closed symbols (,▪) are in the presence of indicated target plus 50 fmol of the other target. Points in FIG. 5A show the short wavelength-resolved cps summed from 0-12 sec (5×τ_{1/2} of EcoB1932-1947(−) HICS18), and points in FIG. 5B show the long wavelength-resolved cps summed from 12-101 sec (after 5×τ_{1/2} of EcoB1932-1947(−) HICS18 through 5×τ_{1/2} of CalA1185-1206(−) wsHICS88). All conditions were performed at least in duplicate. Solid lines through increasing data points are linear best fits: (A) y=0.960x+4.82 and y=0.914x+4.91 for EcoB1932-1947(−) HICS18 without or with CalA1174-1217(+), respectively; and (B) y=0.768x+4.89 and y=0.788x+4.86 for CalA1185-1206(−) wsHICS88 without or with EcoB1921-1958(+), respectively. Repeated experiments (not shown) yielded similar results: y=0.958x+4.84 and y=0.912x+4.92 for EcoB1932-1947(−) HICS18 without or with CalA1174-1217(+), respectively; and y=0.757x+4.90 and y=0.835x+4.71 for CalA1185-1206(−) wsHICS88 without or with EcoB1921-1958(+), respectively; R^{2≧}0.99 in all cases. Solid lines through horizontal data points are linear best fits to 50 fmol specific target in the presence of increasing (as indicated on x-axis) nonspecific target (slope <0.021 in all cases). The dotted lines intersect with the solid target-response at the minimum of one target that can be distinguished in the presence of 200 fmol of the other target.

### DETAILED DESCRIPTION OF THE INVENTION

For clarity of disclosure, and not by way of limitation, the detailed description of the invention is divided into the subsections that follow.

### A. Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

Approximating language, as used herein throughout the specification and claims, may be applied to modify any quantitative or qualitative representation that could permissibly vary without resulting in a change in the basic function to which it is related. Accordingly, a value modified by a term such as “about” is not to be limited to the precise value specified, and may include values that differ from the specified value.

As used herein, the term “sample” generally refers to anything which may contain the analyte for which an assay is desired, more specifically target nucleic acids. The sample may be a biological sample, such as a biological fluid or a biological tissue. Examples of biological fluids include blood, plasma, serum, saliva, serum, sputum, urine, cerebral spinal fluid, tears, mucus, amniotic fluid, semen, stool, or the like. Biological tissues are aggregate of cells, usually of a particular kind of together with their intercellular substance that form one of the structural materials of a human structure, including connective, epithelium, muscle and nerve tissues. Examples of biological tissues also include organs, tumors, lymph nodes, arteries and individual cells.

As used herein, the term “nucleic acid” refers to a polynucleotide compound, which includes oligonucleotides, comprising nucleosides or nucleoside analogs that have nitrogenous heterocyclic bases or base analogs, covalently linked by standard phosphodiester bonds or other linkages. Nucleic acids include RNA, DNA, chimeric DNA-RNA polymers or analogs thereof. In a nucleic acid, the backbone may be made up of a variety of linkages, including one or more of sugar-phosphodiester linkages, peptide-nucleic acid (PNA) linkages (PCT Pub No. WO 95/32305), phosphorothioate linkages, methylphosphonate linkages, or combinations thereof. Sugar moieties in a nucleic acid may be ribose, deoxyribose, or similar compounds with substitutions, e.g., 2′ methoxy and 2′ halide (e.g., 2′-F) substitutions. Nitrogenous bases may be conventional bases (A, G, C, T, U), analogs thereof (e.g., inosine; The Biochemistry of the Nucleic Acids 5-36, Adams et al., ed., 11th ed., 1992), derivatives of purine or pyrimidine bases (e.g., N4-methyl deoxygaunosine, deaza- or aza-purines, deaza- or aza-pyrimidines, pyrimidines or purines with altered or replacement substituent groups at any of a variety of chemical positions, e.g., 2-amino-6-methylaminopurine, O6-methylguanine, 4-thio-pyrimidines, 4-amino-pyrimidines, 4-dimethylhydrazine-pyrimidines, and O4-alkyl-pyrimidines, or pyrazolo-compounds, such as unsubstituted or 3-substituted pyrazolo[3,4-d]pyrimidine (e.g., U.S. Pat. Nos. 5,378,825, 6,949,367 and PCT Pub. No. WO 93/13121)). Nucleic acids may include “abasic” positions in which the backbone does not have a nitrogenous base at one or more locations (U.S. Pat. No. 5,585,481), e.g., one or more abasic positions may form a linker region that joins separate oligonucleotide sequences together. A nucleic acid may comprise only conventional sugars, bases, and linkages as found in conventional RNA and DNA, or may include conventional components and substitutions (e.g., conventional bases linked by a 2′ methoxy backbone, or a polymer containing a mixture of conventional bases and one or more analogs). The term includes “locked nucleic acids” (LNA), which contain one or more LNA nucleotide monomers with a bicyclic furanose unit locked in a RNA mimicking sugar conformation, which enhances hybridization affinity for complementary sequences in ssRNA, ssDNA, or dsDNA (Vester et al., 2004, Biochemistry 43(42):13233-41).

As used herein, the interchangeable term “oligonucleotide” refers to nucleic acid polymers generally made of less than 1,000 nucleotide (nt), including those in a size range having a lower limit of about 2 to 5 nt and an upper limit of about 500 to 900 nt. Preferred oligonucleotides are in a size range having a 5 to 15 nt lower limit and a 50 to 500 nt upper limit, and particularly preferred embodiments are in a size range having a 10 to 20 nt lower limit and a 25 to 150 nt upper limit. Preferred oligonucleotides are made synthetically by using any well-known in vitro chemical or enzymatic method, and may be purified after synthesis by using standard methods, e.g., high-performance liquid chromatography (HPLC). Representative oligonucleotides discussed herein include detection probe oligonucleotides, such as stem-loop oligonucleotides (e.g., oligonucleotides forming MB and HICS probes).

As used herein, the term “label” generally refers to a molecular moiety or compound that can be detected or lead to a detectable response, which may be joined directly or indirectly to a nucleic acid probe. Direct labeling may use bonds or interactions to link label and probe, which includes covalent bonds, non-covalent interactions (hydrogen bonds, hydrophobic and ionic interactions), or chelates or coordination complexes. Indirect labeling may use a bridging moiety or linker (e.g. antibody, oligonucleotide, or other compound), which is directly or indirectly labeled, which may amplify a signal. Preferred labels include a detectable chemiluminescent moiety. Preferred chemiluminescent labels include acridinium ester (“AE”) and derivatives thereof (U.S. Pat. Nos. 5,639,604, 5,656,207 and 5,658,737). Preferred labels are detectable in a homogeneous assay in which bound labeled probe in a mixture exhibits a detectable change compared to that of unbound labeled probe, e.g., stability or differential degradation, without requiring physical separation of bound from unbound forms (e.g., U.S. Pat. Nos. 5,283,174, 5,656,207 and 5,658,737). General methods of synthesizing labels, attaching labels to nucleic acids, and detecting labels are well known (e.g., Sambrook et al., Molecular Cloning, A Laboratory Manual, 2nd ed. (Cold Spring Harbor Laboratory Press, Cold Spring Harbor, N.Y., 1989), Chapter 10; U.S. Pat. Nos. 4,581,333, 5,283,174, 5,547,842, 5,656,207 and 5,658,737).

As used herein, “detection” of the target nucleic acids generally relies on probes in which signal production is linked to the presence of the target sequence because a change in signal results only when the labeled probe binds to amplified product, such as in a molecular beacon (MB), molecular torch, hybridization switch, or hybridization-induced chemiluminescence signal (HICS) probe (e.g., U.S. Pat. Nos. 5,118,801, 5,312,728, 5,925,517, 6,150,097, 6,361,945, 6,534,274, 6,835,542, 6,849,412, 7,169,554 and 8,034,554; and U.S. Pub. Nos. 2006/0194240 A1 and 2007/0166759 A1). Preferred probes use a chemiluminescent label attached to one end of the probe and an interacting compound (e.g., quencher) attached to another location of the probe to inhibit signal production from the label when the probe is in a “closed” conformation that indicates it is not hybridized to its target sequence, but a detectable optical signal is produced when the probe is hybridized to the target sequence which changes its conformation to “open”. Detection of an optical signal from directly or indirectly labeled probes that specifically associate with the target sequence indicates the presence of the target nucleic acid.

Further, the terms “detecting,” “assessing” and “measuring” as used herein are intended to include both quantitative and qualitative determination in the sense of obtaining an absolute value for the amount or concentration of the analyte present in the reaction system, and also of obtaining an index, ratio, percentage, visual or other value indicative of the level of analyte in the reaction system. In contrast, the term “quantifying” specifically refers to quantitative determinations.

As used herein, the term “substantially complementary” refers to two nucleic acid sequences that specifically hybridize. The terms encompasses sequences that are perfectly complementary as well those that contain minor regions of mismatch, wherein the total number of mismatched nucleotides is no more than about 3 for a sequence up to about 25 nucleotides in length. “Specific hybridization” is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tm) for the specific sequence at a defined ionic strength and pH. Equations for calculating Tm values and conditions for nucleic acid hybridization are well known in the art.

As used herein, the term “coupled” generally refers to the attachment of atoms or molecules by either a covalent bond or a non-covalent bond (e.g., a hydrogen bond). Further, the term “coupled” as used herein refers to both direct and indirect coupling. Indirect coupling usually involves a linker of an appropriate length to allow efficient energy transfer between an excited donor chemiluminescent moiety and an acceptor luminophore coupled thereto, resulting in the emission of light in the spectral range of the luminophore. The linker may include a linear, branched, or cyclic alkyl, alkenyl, alkynyl, alkoxyl, or aralkyl chain of less than 50 angstroms with up to 20 heteroatoms, preferably less than 30 angstroms with up to 12 heteroatoms, and most preferably less than 10 angstrom with up to 8 heteroatoms. The linker may contain a functional linkage resulting from the coupling of the functionalized side chains of the acridinium nucleus and the functionalized luminophore. Such functional linkages include, but not limited to, the following commonly encountered ones: —NHCO— (amide), —CONH— (amide), —NHCOO— (carbamate), —O— (ether), —C═N—O— (oxime ether), —S— (thioether, or sulfide), —S—S— (disulfide), —NHCO—NH— (urea), —NHCSNH— (thiourea), —C═N— (imino), —NH— (amino), —N═N— (diazo), —COO— (ester), —C═C— (vinyl, alkenyl, or olefinic), —SO₂NH— (sulfonamide), —C≡C— (alkynyl), —OPO₃—, —PO₃—, —OSO₃—, and —SO₃—. For instance, the luminophore and the chemiluminescent moiety may both be attached to the phosphate backbone of an oligonucleotide at a distance permissive for efficient energy transfer between them.

In contrast, the term “attached” refers to the direct attachment of atoms or molecules by either a covalent bond or a non-covalent bond, preferably a covalent bond.

As used herein, the terms “luminescence” and “luminophore” broadly refer to the emission of light by a substance for any reason other than heat, or a rise in temperature. In most cases, the term “luminescence” refers to fluorescence or chemiluminescence.

The terms “fluorescence” and “fluorophore” as used herein refer to a physicochemical process and a molecular entity, respectively, wherein a molecule emits light of a certain wavelength after photochemical excitation with light of a different wavelength. A “fluorophore” may include, but is not limited to, a dye, intrinsically fluorescent protein and a lanthanide phosphor. Dyes, for example, include rhodamine and derivatives, such as Texas Red, ROX (6-carboxy-X-rhodamine), rhodamine-NHS, and TAMRA (5/6-carboxytetramethyl rhodamine NHS); fluorescein and derivatives, such as 5-bromomethyl fluorescein and FAM (5′-carboxyfluorescein NHS); cyanine dyes and derivatives, such as Cy2, Cy3, Cy3B, Cy3.5, Cy5, Cy5.5 and Cy7; Lucifer Yellow, IAEDANS (5-({2-[(iodoacetyl)amino]ethyl}amino)naphthalene-1-sulfonic acid), 7-dimethyl-N-coumarin-4-acetate, 7-hydroxy-4-methylcoumarin-3-acetate, 7-NH2-4-methylcoumarin-3-acetate (AMCA), monobromobimane, pyrene trisulfonates, such as Cascade Blue, and monobromotrimethyl-ammoniobimane.

The terms “chemiluminescence” and “chemiluminophore” as used herein refer to a physicochemical process and a molecular entity, respectively, wherein a molecule emits light after a chemical initiation reaction, which is also referred to herein as a chemical trigger. Acridinium compounds such as acridinium sulfonamides and acridinium esters (AE) are common examples of chemiluminescent compounds that have been disclosed for use in the type of molecular detection assay described herein.

As used herein, the term “quencher” refers to a chromophoric molecule or part of a compound, which is capable of reducing the emission from a luminescent donor when attached to or in proximity to the donor. Luminescence is “quenched” when the luminescence emitted by the luminophore is reduced as compared with the luminescence in the absence of the quencher by at least 10%, for example, 15%, 20%, 30%, 40%, 50%, 60%, 70%, 80%, 90%, 95%, 98%, 99%, 99.9% or more. There is a great deal of practical guidance available in the literature for selecting appropriate reporter-quencher pairs for particular probes (e.g., Clegg, Proc. Natl. Acad. Sci. USA, 1993, 90:2994-98; Wu et al., Anal. Biochem., 1994, 218:1-13; Pesce et al., eds., FLUORESCENCE SPECTROSCOPY, 1971, Marcel Dekker, New York; White et al., FLUORESCENCE ANALYSIS: A PRACTICAL APPROACH, 1970, Marcel Dekker, New York). A number of commercially available quenchers are known in the art, and include but are not limited to Dabcyl, BHQ-1, BHQ-2, and BHQ-3. In addition, these quenchers have no native fluorescence, virtually eliminating background problems seen with other quenchers. BHQ quenchers can be used to quench almost all reporter detectable labels and are commercially available, for example, from Biosearch Technologies, Inc. (Novato, Calif.). Suitable linking methodologies for attachment of many detectable labels to oligonucleotides are described in many references (e.g., Marshall, Histochemical J., 1975, 7:299-303; U.S. Pat. No. 5,188,934).

### B. Wavelength-Shifted Chemiluminescence Probes

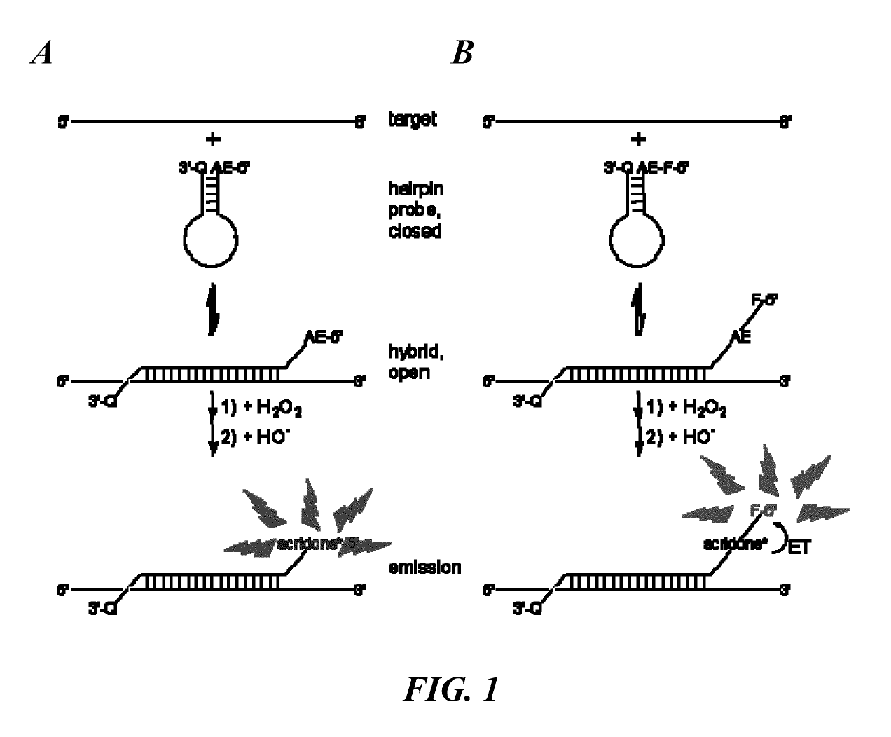

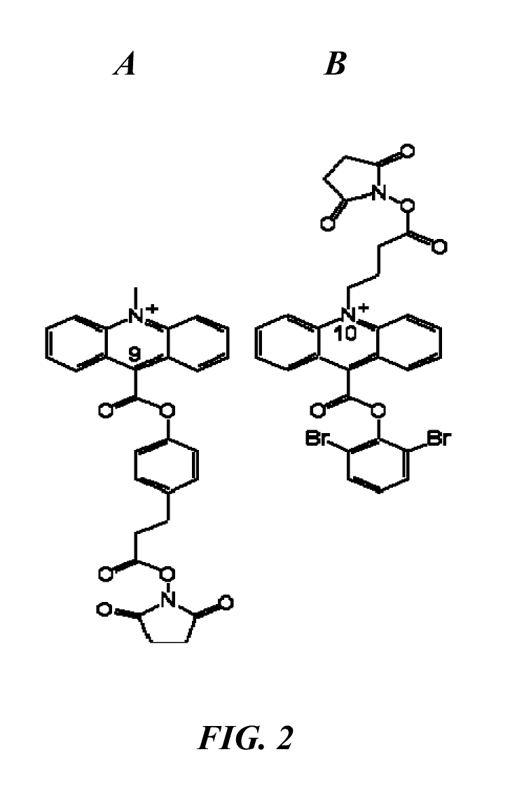

Hybridization-induced chemiluminescent signal (HICS) probes have been described recently (U.S. Pat. No. 7,169,554; Brown et al., Org. Biomol. Chem. 2009, 7:386-94). These probes comprise structures similar to molecular beacons except that they possess an AE chemiluminophore instead of a fluorophore attached to the terminus opposite the quenching moiety (FIG. 1A). An important aspect of their design is the use of an AE label attached to the probe sequence through the N10 position of the acridinium rather than through groups linked to the C9 position (FIG. 2). This enables the excited 10-alkyl-9(10H)-acridone (N-alkylacridone*) to remain attached to the probe sequence and in proximity to other chromophores on the same probe sequence during detection (Browne et al., J. Am. Chem. Soc. 2011, 133:14637-48). A further design characteristic is the facilitation of rapid chemiluminescence initiation at a sufficiently low pH≦9 to maintain hybridization between the probe arms or between the probe loop region and the target in the absence or presence of complementary target sequence, respectively. This has been previously studied in terms of the reduction of the leaving group pKₐ through substitution of the phenoxy group (U.S. Pat. No. 5,756,709; Smith et al., J. Photochem. Photobiol. A: Chem. 2000, 132:181-91; and Smith et al., J. Photochem. Photobiol. A: Chem. 2009, 203:72-79). Not only did di-ortho-bromo substitution (pKₐ ˜6.4; cf pKₐ ˜9.9 and 10.6 for unsubstituted phenol and 2,6-dimethylphenol, respectively) increase the rate of chemiluminescence emission compared to the unsubstituted phenol under the same detection conditions, but it also increased the storage stability relative to the unsubstituted phenol. An important stability aspect of the reagent formulation for assays using HICS probes includes use of a low pH buffer and surfactant in the hybridization reagent to provide conditions that protect the acridinium esters from hydrolysis and nonspecific adsorption and nucleic acids from nucleases. Additionally, inclusion of an appropriate surfactant enhances the chemiluminescence yield from the luminophore of acridinium esters (Bagazgoitia et al., J. Biolum. Chemilum. 1988, 2:121-28; Natrajan et al., Org. Biomol. Chem. 2011, 9:5092-5103).

Excitation of different emitter fluorophores by a single type of nearby harvester fluorophore species has been demonstrated with molecular beacons complementary to different target sequences (Tyagi et al., Nat. Biotechnol. 2000, 18:1191-96). Here we demonstrate that chemical initiation of a single type of AE label on a HICS probe (analogous to a harvester fluorophore) can provide sufficient energy to transfer to different proximally-attached emitter fluorophores, enabling light emission at wavelengths distinct from AE itself (FIG. 1B).

Accordingly, the first aspect of the invention relates to a chemiluminescent probe for detecting a target nucleic acid sequence in a sample, e.g., a biological or environmental sample. The probe includes at least the following structural elements: (1) a target binding region with a base sequence that is substantially complementary to a portion of the target nucleic acid sequence; (2) an acridinium ester (AE) moiety attached to a first region flanking the target binding region; (3) a luminophore coupled to the AE moiety to allow energy transfer from an acridone moiety, produced by a chemical triggering of the AE moiety, to the luminophore; and (4) a quencher moiety attached to a second region flanking the target binding region, such that the first and second flanking regions are on the opposite sides of the target binding region. In the absence of the target nucleic acid sequence, the probe is in an inactive conformation, wherein the quencher moiety is sufficiently close to the AE-coupled luminophore to substantially block its emission. In the presence of the target nucleic acid sequence, the probe assumes an active conformation, wherein the quencher moiety moves sufficiently far from the AE-coupled luminophore so that emission from the luminophore can be detected following the chemical triggering of the AE moiety.

Thus, each chemiluminescent probe according to the present invention must incorporate at least five features: (i) an oligonucleotide strand complementary to an oligonucleotide sequence in a particular target nucleic acid marker; (ii) extensions on either side of the aforesaid oligonucleotide sequence by oligonucleotide strands (stem arms) complementary to each other so that they cause the two ends of the oligonucleotide to bind to each other in a hairpin-like arrangement; (iii) a chemiluminescent AE moiety attached to one end of the oligonucleotide sequence via an aminoalkyl group or related spacer; (iv) a luminophore coupled to the AE moiety to permit energy transfer; and (v) a quencher for the luminescence emitted by the AE chemiluminophore or AE-coupled luminophore, attached to the other end of the oligonucleotide sequence via an appropriate spacer (see FIG. 1). The stem arms may be partially or completely complementary to the target sequence. When the stem arms hybridize to themselves in the absence of target, the target complementary sequence typically forms a loop structure.

In some embodiments, the secondary structure of the chemiluminescent probe corresponds to that of a molecular beacon, molecular torch, hybridization switch, or HICS probe. In some embodiments, the AE moiety is conjugated to a site proximal to a terminus of a stem-loop oligonucleotide, and a quenching moiety is attached proximal to the opposite terminus of the oligonucleotide. Alternatively, the AE and quenching moieties may be conjugated to sites that are proximal to the stem-loop junction or at other positions on the stem of a stem-loop oligonucleotide. In some embodiments, the AE moiety is conjugated an acridinium position other than C9 to a stem-loop oligonucleotide. For example, the AE moiety may be conjugated through the N10 acridinium position (N-linked) to a stem-loop oligonucleotide. Preferably, the AE moiety is a 9-(2,6-dibromophenoxycarbonyl)-10-(3-carbonylpropyl)acridinium salt (e.g., iodide). In some embodiments, the luminophore is a fluorescent dye, e.g., a fluorescein dye, a rhodamine dye, or a cyanine dye. The luminophore may be coupled to the AE moiety directly or via a linker. Preferably, the luminophore is attached to the oligonucleotide backbone at or near the first region flanking the target binding region, so that the AE moiety and the luminophore are linked to each other by a small oligonucleotide fragment of the probe. The quencher moiety may include a wide variety of molecules capable of blocking emission from the AE-coupled luminophore. Without limitation, examples of such molecules include Dabcyl and Black Hole Quencher 2 (BHQ-2).

As noted above, one of the objects of the present invention is to provide an improved method for detecting and quantifying multiple nucleic acid targets in the same sample. The target nucleic acid sequences can be from a mammalian (e.g., human) organism, a bacterium, a fungus, a virus, or any other organism of interest. For example, simultaneous quantitative determination of multiple infectious organisms represents one attractive application of the present invention. In some embodiments, the target nucleic acid sequences are selected from the group consisting of an Enterococcus target sequence, a pan-fungal target sequence, and a Neisseria gonorrhoeae (N. gonorrhoeae) target sequence. Selected target and probe sequences are set forth in Table 1.

TABLE 1
Selected target and probe nucleic acid sequences.

| SEQ ID | Nucleotide Sequence(5′ → 3′) | Length |
|---|---|---|
| 1 | TTAGGGCTAG CCTCGGAATT GAGAATGATG G | 31 |
| 2 | GUCCUAAGGU AGCGAAAUUC CUUGUCGGGU AAGUUCCG | 38 |
| 3 | GACUCAACAC GGGGAAACUC ACCAGGUCCA GACACAAUAA GG | 42 |
| 4 | UAACCGUUCU CAUCGCUCUA CGGA | 24 |
| 5 | UCCCCCGCUA CCCGGUACGU UCC | 23 |
| 6 | CTCAATTCCG AGGCT | 15 |
| 7 | CGACAAGGAA UUUCGC | 16 |
| 8 | GTCTGGACCT GGTGAGTTTC CC | 22 |
| 9 | AGAGCGAUGA GAAC | 14 |
| 10 | GUACCGGGUA GCG | 13 |
| 11 | TGCGTGCTCA ATTCCGAGGC TCACGCA | 27 |
| 12 | TTGCGTGCTC AATTCCGAGG CTCACGCA | 28 |
| 13 | CCCAGCACCG ACAAGGAAUU UCGCGTGCTG GG | 32 |
| 14 | CCGAGGACGT CTGGACCTGG TGAGTTTCCC GTCCTCGG | 38 |
| 15 | CCGACAGAGC GAUGAGAACG UCGG | 24 |
| 16 | CCGAGGUACC GGGUAGCGCU CGG | 23 |
| 17 | UAACCGUUCU CAUCGCUCUA CGGAC | 25 |
| 18 | UCCCCCGCUA CCCGGUACGU UCCC | 24 |

The Enterococcus target nucleic acid sequence may include a fragment of Enterococcus faecalis (E. faecalis) 23S rRNA corresponding to nucleotides 866-896 and consisting of SEQ ID NO:1, allowing for a DNA equivalent thereof. In some embodiments, an Enterococcus probe may include a stem-loop oligonucleotide having a target complementary base sequence consisting of SEQ ID NO:6, allowing for an RNA equivalent thereof. In preferred embodiments, the stem-loop oligonucleotide may include a base sequence consisting of any one of SEQ ID NOs:11 and 12, allowing for RNA equivalents thereof.

The pan-fungal target nucleic acid sequence may include a fragment of Candida albicans (C. albicans) 18S rRNA corresponding to nucleotides 1174-1217 and consisting of SEQ ID NO:3, allowing for a DNA equivalent thereof. In some embodiments, a pan-fungal probe may include a stem-loop oligonucleotide having a target complementary sequence consisting of SEQ ID NO:8, allowing for an RNA equivalent thereof. In preferred embodiments, the stem-loop oligonucleotide may include a base sequence consisting of SEQ ID NO:14, allowing for an RNA equivalent thereof.

The N. gonorrhoeae target nucleic acid sequence may include a 16S rRNA fragment corresponding to nucleotides 128-150 and consisting of SEQ ID NO:5, allowing for a DNA equivalent thereof. In some embodiments, an N. gonorrhoeae probe may include a stem-loop oligonucleotide having a target complementary base sequence consisting of SEQ ID NO:10, allowing for a DNA equivalent thereof. In preferred embodiments, the stem-loop oligonucleotide may include a base sequence consisting of SEQ ID NO:16, allowing for a DNA equivalent thereof.

In some embodiments, the chemiluminescent probes of the present invention provide a detection range of up to about 3 log units, preferably from about 200 amol to about 200 fmol, from about 500 amol to about 200 fmol, from about 2 fmol to about 200 fmol, from about 500 amol to about 50 fmol, or from about 500 amol to about 5 fmol. Multiple wavelength-shifted probes are capable of detecting two, three, four, five, or more different target sequences, provided the probes' emission profiles are sufficiently distinct to permit spectral and/or temporal resolution.

### C. Kits for Chemiluminescent Detection

As noted above, the present invention also provides kits for detecting and/or quantifying a target nucleic acid sequence in a sample that includes a wavelength-shifted chemiluminescent probe according to the present invention and reagent means for triggering a chemiluminescence reaction. In some embodiments, the kit may detect and/or quantify a plurality of target nucleic acid sequences in a sample. Such a kit preferably contains reagent means triggering a chemiluminescence reaction and a plurality of wavelength-shifted chemiluminescent probes having sufficiently different emission profiles to allow spectral and/or temporal resolution of their luminescence emissions. The oligonucleotide structure of the chemiluminescent probe preferably corresponds to that of a molecular beacon, molecular torch, hybridization switch, or hybridization-induced chemiluminescence signal (HICS) probe (e.g., U.S. Pat. Nos. 5,118,801, 5,312,728, 5,925,517, 6,150,097, 6,361,945, 6,534,274, 6,835,542, 6,849,412, 7,169,554 and 8,034,554; and U.S. Pub. Nos. 2006/0194240 A1 and 2007/0166759 A1). In some embodiments, the plurality of wavelength-shifted probes may include different luminophores coupled to the same AE moiety. Alternatively, different AE moieties may also be employed. In some embodiments, the AE moiety is conjugated an acridinium position other than C9 to a stem-loop oligonucleotide. For example, the AE moiety may be conjugated through the N10 acridinium position (N-linked) to a stem-loop oligonucleotide. Preferably, the AE moiety is a 9-(2,6-dibromophenoxycarbonyl)-10-(3-carbonylpropyl)acridinium salt (e.g., iodide). In some embodiments, the luminophore is a fluorescent dye, e.g., a fluorescein dye, a rhodamine dye, or a cyanine dye. The quencher moiety may include a wide variety of molecules capable of blocking emission from the AE-coupled luminophore. Without limitation, examples of such molecules include Dabcyl and Black Hole Quencher 2 (BHQ-2).

The present invention provides an improved route to rapid synthesis of sensitive, wavelength-distinguishable, chemiluminescent probe sets that can be individually detected and quantified simultaneously after a chemical initiation step. Chemiluminescent outputs from several wavelengths have been successfully achieved based on energy transfer from a chemiluminophore (AE) to different luminophores (e.g., fluorophores) covalently linked various distances from the chemiluminophore, with emissions defined and characteristic of the coupled luminophores only, thus permitting rational design of probe labels for multiplex end-point molecular assays. The self-quenching nature of these nucleic acid probes in the absence of complementary target ensures they are amenable to homogeneous hybridization assays for detection and/or quantification of any desired target. The invention can be better understood by reference to following non-limiting examples.

### EXAMPLES

### Example 1

### Selection of Target and Probe Sequences

Five target sequences were selected (Table 1: SEQ ID NOs:1-5, 17, 18; Table 2) as representatives of two different, broad microbial groups found in environmental samples such as seawater (Enterococcus faecalis, Escherichia coli, and Candida albicans) (Hartz et al., J. Environ. Qual. 2008, 37:898-905; Papadakis et al., Water Res. 1997, 31:799-804) or of two pathogens that may occur individually or simultaneously in human urogenital swab or urine specimens (Chlamydia trachomatis and Neisseria gonorrhoeae) (Johnson et al., Clin. Chem. 2001, 47:760-63). In order to mimic more closely the natural nucleic acid materials, the synthetic target oligonucleotide sequences exceeded the length of the part for which complementary sequences in probes would be constructed (Table 1: SEQ ID NOs:6-10; Table 2: central part in each target sequence). These targets were synthesized on automated oligonucleotide synthesizers by established methods using phosphoramidite monomers, then purified and quantified prior to use, as described in greater detail in Example 2. Target sequences denoted as CtrB1447-1470(−) and NgoA128-150(−) in Table 2 contain a 3′-terminal deoxycytidine residue with a reversed 3′→5′ polarity and directly correspond to SEQ ID NOs:17 and 18 in Table 1. SEQ ID NOs: 4 and 5 do not contain this residue because it is not believed to play a role in target-probe hybridization.

Selected probe sequences (Table 1: SEQ ID NOs: 11-16; Table 2) were based on earlier fluorescent probes (Browne, K. A., J. Am. Chem. Soc. 2005, 127:1989-94) used in detection of pan-bacterial and pan-fungal species or were derived from earlier probe sequences (U.S. Pat. No. 5,693,468) used to detect C. trachomatis and N. gonorrhoeae specifically but with self-complementary arm sequences added to each pair of termini. The probes were also synthesized on automated oligonucleotide synthesizers by established methods using phosphoramidite monomers. Several different pairs of stem arms were incorporated into the oligonucleotide sequences, all of which included GC clamps at their termini to maximize the affinity of the stem arms for each other. The probe sequences were furnished with one of two quenching moieties: a 3′-Dabcyl or a 3′-BHQ2 unit incorporated as part of the oligonucleotide synthesis (U.S. Pat. No. 7,169,554; U.S. Pub. No. 2007/0166759 A1; Brown, supra). 5′-Fluorophore moieties were coupled to the probe oligonucleotides during automated synthesis as described in detail in Example 2. The probe oligonucleotides were synthesized with a terminal protected 5′-hexylamino phosphoramidite at the end of the oligonucleotide synthesis sequence. After deprotection, AE moiety was used to label the freed 5′-NH₂ group as described in detail in Example 2. The probes produced were purified and quantified prior to use.

In Table 2, target and corresponding probe names derived from the three letter abbreviations (Genus species) of the reference sequences, a letter indicating the small (A) or large (B) rRNA subunit, the target sequence range and an internally-derived unique designation for the probes (e.g., HICS1). Efa sequences are Enterococcus specific probes and targets to/from the Enterococcus faecalis 23S rRNA reference sequence (NCBI Accession No. AJ295306). Eco sequences are pan-bacterial probes and targets to/from the Escherichia coli 0157:H7 23S rRNA reference sequence (NCBI Accession No. E16366). Cal sequences are pan-fungal probes and targets to/from the Candida albicans 18S rRNA reference sequence (NCBI Accession No. E15168). Ctr and Ngo sequences are probes and targets to/from Transcription-Mediated Amplification amplicon sequences of Chlamydia trachomatis 23S rRNA (NCBI Accession No. AM884176) and Neisseria gonorrhoeae 16S rRNA (NCBI Accession No. NC 002946), respectively. Target names are given in bold characters. Numbering to reference targets indicated underneath each class of reference sequences. Backbone sugar structures of probe and target nucleic acids: DNA, deoxyribose (underlined); RNA, ribose; OMe, 2′-methoxyribose. OMe oligonucleotides have similar affinities for complementary RNA sequences (Lesnik et al., Biochemistry 1998, 37:6991-97) while being more resistant to nucleases than RNA oligonucleotides (Sproat, et al., Nucleic Acids Res. 1989, 17:3373-86). AE signifies the position of an N-linked AE chemiluminophore label on an amine linker. FAM, TAM and Cy5 denote the attached fluorophores 6-carboxyfluorescein, 5- and 6-carboxytetramethylrhodamine and cyanine dye 5 labels, respectively. D and BHQ2 indicate the attachment location of the quenching moieties Dabcyl and Black Hole Quencher-2.

TABLE 2
Nucleic acid sequences for chosen targets and the probes developed from them.

| | Nucleic acid | |
|---|---|---|
| Name | backbone | Sequence and substituents |
| aB874-888(−) HICS1 | DNA | 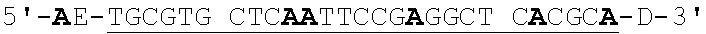 |
| EfaB874-888(−) wsHICS10a | DNA | 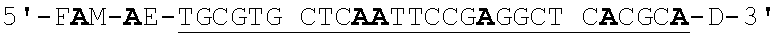 |
| EfaB874-888(−) wsHICS10b | DNA | 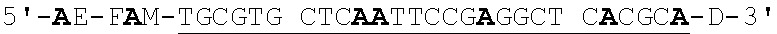 |
| EfaB874-888(−) wsHICS11a | DNA | 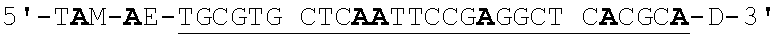 |
| EfaB874-888(−) wsHICS12a | DNA | 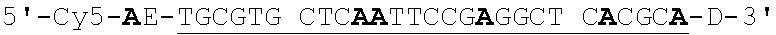 |
| EfaB874-888(−) wsHICS20a | DNA | 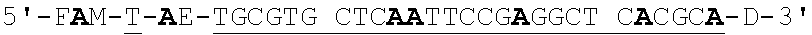 |
| EfaB866-896(+) | DNA | 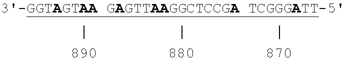 |
| EcoB1932-1947(−) HICS18 | OMe/DNA | 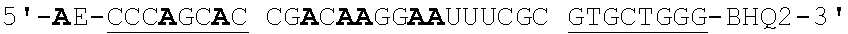 |
| EcoB1921-1958(+) | RNA | 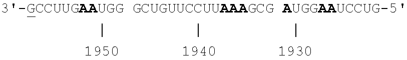 |
| CalA1185-1206(−) wsHICS88 | DNA | 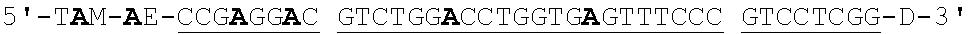 |
| CalA1174-1217(+) | RNA | 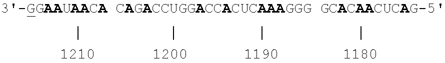 |
| CtrB1452-1465(+) HICS51 | OMe | 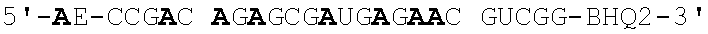 |
| CtrB1447-1470(−) | OMe | 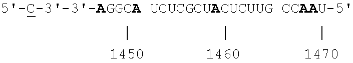 |
| CtrB1452-1465(+) HICS51 | OMe | 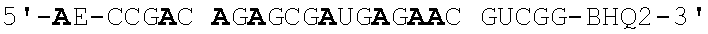 |
| CtrB1447-1470(−) | OMe | 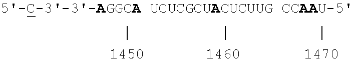 |
| NgoA133-145(+) wsHICS63 | OMe | 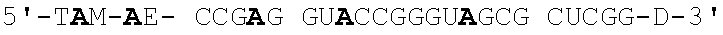 |
| NgoA128-150(−) | OMe | 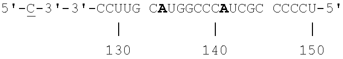 |

### Example 2

### Synthesis and Labeling of Oligonucleotides

Expedite model 8909 Nucleic Acid Synthesis Systems (PerSeptive Biosystems, now part of Life Technologies Corporation, Carlsbad, Calif.) were used to synthesize oligonucleotides on substituted 500 Å controlled pore glass (CPG) substrates packed in automated synthesizer columns. Deoxy CPG (Proligo, now part of Sigma-Aldrich, Boulder, Colo.) were used for DNA and RNA target syntheses, resulting in 3′-deoxyribonucleotides on these oligonucleotides. Probe sequences incorporating quencher units started with Black Hole Quencher-2 attached to a CPG via a glycolate linker and with a dimethoxytrityl (DMT) group protecting the terminal hydroxyl (p/n CG5-5042G, Biosearch Technologies, Inc., Novato, Calif.) or with a protected Dabcyl attached to a CPG (1-dimethoxytrityloxy-3[O-(N-4′-carboxy-4-(dimethylamino)-azobenzene)-3-aminopropyl)]-propyl-2-O-succinoyl-long chain alkylamino-CPG, p/n CPG1002N12DABXS, 3 Prime (a division of Prime Synthesis, Inc., Aston, Pa.). 3′-β-Cyanoethyl (3′-CE) phosphoramidites for RNA and 2′-OMeRNA were from Pierce Biotechnology (now part of Thermo-Fisher Scientific Inc., Rockford, Ill.), and 3′-CE phosphoramidites for DNA were from Proligo. Fluorophore moieties were coupled to the probe oligonucleotides during automated synthesis by using a 6-carboxyfluorescein hexylamino CE phosphoramidite (5′-FAM, p/n BGX-3008, BioGenex Laboratories Inc., Fremont, Calif.), 2-dimethoxytrityloxymethyl-6-(3′,6′-dipivaloylfluorescein-6-yl)-hexyl-1-O-(2-cyanoethyl)-(N,N-diisopropyl)-phosphoramidite (internucleotidyl FAM, p/n 10-1964, Glen Research, Sterling, Va.), 6-carboxytetramethylrhodamine piperidinyl CE phosphoramidite (5′-TAM, p/n BNS-5027B, Biosearch Technologies, Inc.) or 1-[3-(4-monomethoxytrityloxy)propyl]-1′-[3-[(2-cyanoethyl)-(N,N-diisopropyl phosphoramidityl]propyl]-3,3,3′,3′-tetramethylindodicarbocyanine chloride (5′-Cy5, p/n 10-5915, Glen Research). 3-Dimethoxytrityloxy-1-(6-(fluorenylmethoxycarbonylamino) hexanamido)propyl-2-O-(2-cyanoethyl)-(N,N-diisopropyl)-phosphoramidite (U.S. Pat. No. 5,585,481; p/n 29-0036, Thermo Fisher Scientific Inc., Waltham, Mass.) and 6-(4-monomethoxytritylamino)hexyl (2-cyanoethyl) (N,N-diisopropyl) phosphoramidite (p/n 10-1906, Glen Research) were used to functionalize probe oligonucleotides with internal and 5′-terminal amine linker arms, respectively. Completed oligonucleotides were hydrolyzed from the CPG column with ammonium hydroxide (33%, 60° C., ≧16 h), followed by purification by polyacrylamide gel electrophoretic separation {15% polyacrylamide (19:1 acrylamide:bisacrylamide), 7 M urea, 1× TBE buffer (89 mM Tris base, 89 mM boric acid, 2 mM ethylenediamine-N,N,N′N′-tetraacetic acid (EDTA), pH 8.2)}, and desalted with Sep-Pak Plus C18, 55-105 μm cartridges (p/n WAT020515, Waters Corp., Milford, Mass.), and subsequent precipitation (0.3 M sodium acetate, 75% ethanol, −78° C. (dry ice bath), 10 min).

The monomethoxytrityl (MMT) protecting groups of the 5′-amino moieties were removed by treatment with acetic acid (80%, ˜20° C., 30 min) while the fluorenylmethyl-oxycarbonyl (FMOC) protecting groups of the internucleotidyl aminos were cleaved during hydrolysis of the oligonucleotides from the CPG columns. The amino groups were then labeled through the succinimidyl group of the N-linked AE NHS ester (942,6-dibromophenoxycarbonyl)-10-(3-succinimidyloxycarbonylpropyl)acridinium iodide; FIG. 2B) as described previously (U.S. Pat. No. 7,169,554; Brown, supra). The fully labeled oligonucleotides were sequentially purified by reversed-phase HPLC (System Gold, Beckman Coulter Inc., Brea, Calif.): Jupiter 10 μm C4 300 Å, 250 mm×4 mm column (p/n 00G-4168-E0, Phenomenex, Torrance, Calif.), linear gradient of 5-40% acetonitrile with 100 mM aqueous triethylammonium acetate (pH 7) at 0.5 mL min⁻¹ over 70 min followed by desalting through NAP-10 Sephadex G-25 columns (p/n 17-0854, GE Healthcare, Piscataway, N.J.). The oligonucleotide concentrations were determined by absorption spectroscopy (DU 640B Spectrophotometer, Beckman Coulter, Inc.) in quartz cuvettes from aliquots diluted in water.

### Example 3

### Performance of Wavelength-Shifted HICS Probes

### 3(a) Chemiluminescent Spectrographic Emissions

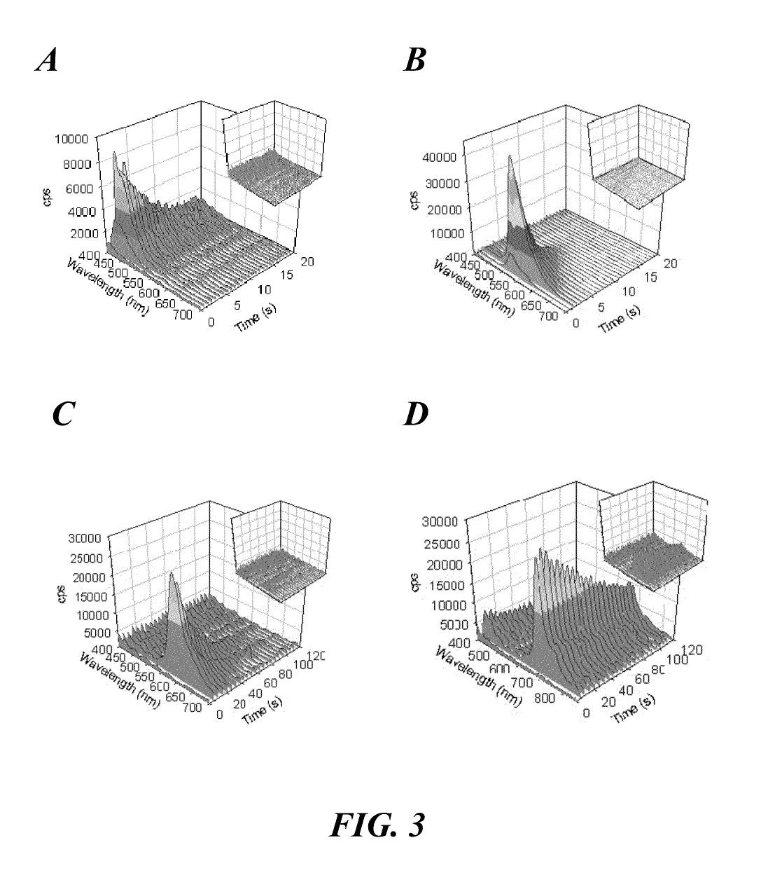

Spectrography was used to record time-resolved spectrograms of chemiluminescence and ET from AE to nearby fluorophores of a series of HICS and wsHICS probes with the same sequence but differing in fluorophore acceptor or its position. Time-resolved, spectrographic images of HICS probe chemiluminescent emissions were acquired on a low light, echelle-type SE200 spectrograph (Optomechanics Research Inc., Vail, Ariz.) using KestrelSpec software (Catalina Scientific Corp., Tucson, Ariz.). The images were transformed into spectrograms at 1 nm resolution and then processed with a locally weighted scatter plot smoothing (4%) algorithm (Cleveland, W. S. J. Am. Stat. Assoc. 1979, 74:829-36; Cleveland, W. S. & Devlin, S. J. J. Am. Stat. Assoc. 1988, 83:596-610) (LOESS utility Excel Add-In, Peltier Technical Services). 50-200 pmol/100 μL of HICS or wsHICS probes, with or without equimolar amounts of complementary synthetic targets (Table 2), were allowed to incubate for 15 min at 60° C. in a low pH, surfactant-containing 1× hybridization reagent (indicating hybridization or pre-detection concentrations; 95 mM succinic acid, 1.5 mM EDTA, 1.5 mM ethylene glycol-bis(2-aminoethylether)-N,N,N′N′-tetraacetic acid (EGTA), 312 mM lithium dodecyl sulfate, 125 mM LiOH, pH=5.2) and cooled for at least 15 min at room temperature. These conditions permitted hybridization of the probe loop regions to target sequences to occur in the solutions containing target, and hybridization of the probe arms to occur in solutions without target. Chemiluminescence from 100 μL of each of these solutions was initiated by a single 100 μL injection of a 240 mM H₂O₂/2 M Tris-HCl pH 9.0 solution, and data collection commenced immediately (25×0.4 sec or 5 sec intervals, depending on the emission durations, separated by 0.37 sec pauses). Results of these experiments are presented in FIG. 3.

In the absence of an attached fluorophore, time-resolved chemiluminescence spectrograms of EfaB874-888(−) HICS 1 plus complementary target quickly increased to a peak before decaying over about 20 sec (FIG. 3A). The sequential spectrograms clearly show emissions in the 400-520 nm wavelength range and double peaks at ˜425 and ˜448 nm that are characteristic of N-alkylacridone* fluorescence emission (McCapra et al., Photochem. Photobiol. 1965, 4:1111-21). In the absence of target (FIG. 3A, inset), chemiluminescence was similar to that from hybridization reagent alone (data not shown), demonstrating efficient quenching. Chemiluminescence from EfaB874-888(−) wsHICS10a plus target also quickly rose to a peak before decreasing over about 10 sec (FIG. 3B). Emission wavelengths ranged from about 490-600 nm with a maximum at about 520 nm, consistent with fluorescein fluorophore emission and suggesting efficient ET from N-alkylacridone* to the fluorophore. Chemiluminescence from EfaB874-888(−) wsHICS11a plus target quickly peaked before slowly decaying over at least 60 sec (FIG. 3C); emissions ranged from about 540-670 nm with a maximum at about 580 nm, as expected from the tetramethylrhodamine fluorophore and again supporting efficient ET from N-alkylacridone* to the fluorophore despite little overlap in N-alkylacridone* emission and tetramethylrhodamine absorption bands (about 460-590 nm). Chemiluminescence from EfaB874-888(−) wsHICS12a plus target quickly increased to a peak but then slowly decayed over more than 120 sec (FIG. 3D). Wavelength emissions ranged from about 600-790 nm with a maximum at about 680 nm, consistent with Cy5 fluorophore emissions (except with an additional peak/shoulder at about 750 nm), supporting efficient ET from N-alkylacridone* to the fluorophore even though there was essentially no overlap between N-alkylacridone* emission and Cy5 absorption wavelengths (about 520-690 nm). The uniform chemiluminescence rise and fall across spectrograms in each example is supportive of detectable emission from single species (N-alkylacridone*, or fluorescein, tetramethylrhodamine or Cy5 fluorophores). These results demonstrate principles to design distinct wsHICS probes based on fluorophore emission characteristics but without the criterion of overlapping chemiluminophore donor emission and fluorophore acceptor absorption wavelengths.

### 3(b) Time-Resolved Chemiluminescence

Constants for total chemiluminescent emissions from the HICS and wsHICS probes complementary to different targets and with different nucleic acid backbones (Table 2) were determined after hybridization to excess complementary target oligonucleotides. Chemiluminescent emission time courses were acquired on a Gen-Probe Incorporated LEADER HC+ Luminometer. Solutions containing 0.5 pmol/100 μL probes with or without 1 pmol/100 μL complementary targets in 1× hybridization reagent plus 100 μL of silicone oil were mixed by vortexing in 12 mm×75 mm polypropylene tubes and allowed to incubate in a 60° C. water bath for 40 min to facilitate hybridization of probes or probes plus target sequences. After the mixtures had cooled to room temperature for at least 10 min, chemiluminescent emissions were initiated by a 200 μL injection of Detect 1 solution (240 mM H₂O₂/1 mM HNO₃) followed by a 2 sec pause and then a 200 μL injection of Detect 2 solution (2 M Tris-HCl pH 9.0). Data collection began after the first 40 ms mixing time and continued for at least 5×τ_{1/2} of the apparent first order rate constant k_{b} (10-500 sec total) without inter-interval delays. Constants were calculated using TableCurve 2D v5.01 (Systat Software, Inc., San Jose, Calif.). Hardware and firmware configurations of the luminometer may limit interpretation of the kinetic constant kₐ. Final pH after detection was always 9.0.

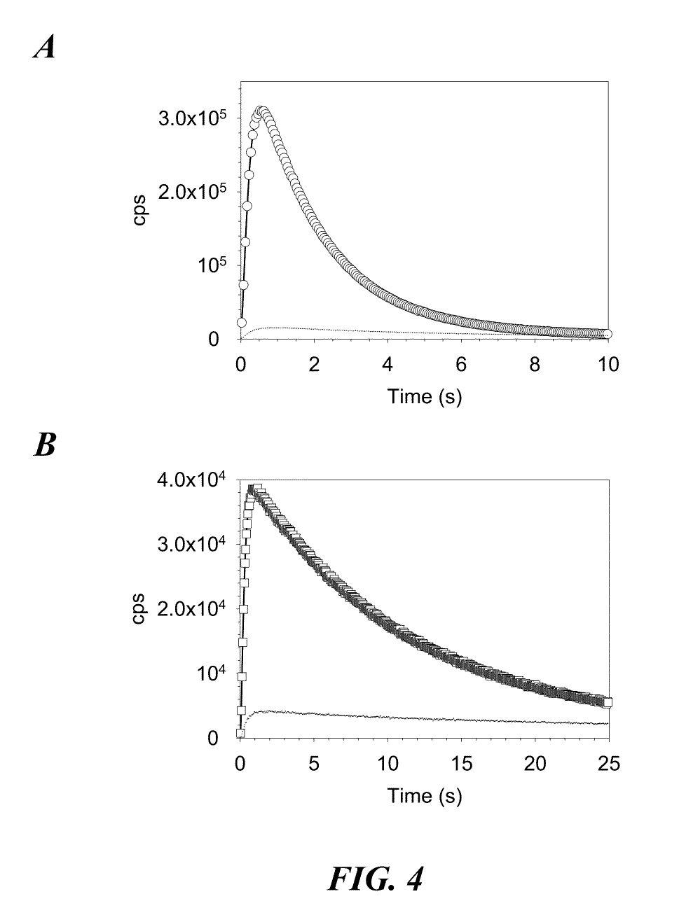

Time-resolved, total wavelength luminescence emissions of the probes were plotted as summed counts per second (cps) versus time (FIG. 4). The emissions increased to a peak then decreased towards zero. As observed previously for chemiluminescent reactions proceeding through an acridone* (Maskiewicz et al., J. Am. Chem. Soc. 1979, 101:5355-64; Browne et al., J. Am. Chem. Soc. 2011, 133:14637-48), two apparent first-order, competing and parallel reactions most simply account for the formation and decay of chemiluminescent emissions. This allowed data fitting to a linear combination of exponential equations (Eq. 1) based on a kinetic scheme (Eq. 2) simplified from one that includes a series of more elementary steps (Browne, supra). A related but alternate set of reaction pathways leading to chemiluminescence from 10-methyl-9-(phenoxycarbonyl)acridinium salts was recently detailed (Krzyminski et al., J. Org. Chem. 2011, 76:1072-85). An important distinction is decomposition of a 1,2-dioxetane intermediate into an N-alkylacridone* and the release of a proposed phenyl carbonate rather than a previously proposed phenolate and CO₂. Distinguishing between these pathways is beyond the scope of the current studies and does not change the currently used simplified kinetic scheme. These optimized fits yielded physical constants that quantitatively defined each of the probes (Table 3), and values back-calculated from Eq. 1 and the physical constants closely followed the observed data points regardless of whether the reaction sequence included energy transfer or not.

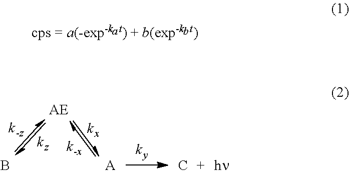

TABLE 3
Chemiluminescent Emission Constants for Probes^{a}

| | Time- | | | | |
|---|---|---|---|---|---|
| | to-peak | | | SA^{c} | |
| Name | (s) | kₐ (s⁻¹)^{b} | k_{b} (s⁻¹)^{b} | (Σcps/pmol) | S/B^{c} |
| EfaB874-888(−) HICS1^{d} | 0.55 [6] | 4.66 ± 0.18 [10] | 0.532 ± 0.017 [10] | (2.99 ± 0.14) × 10⁷ [10] | 11.1 ± 0.5 [10] |
| EfaB874-888(−) wsHICS10a^{d} | 0.58 [4] | 3.07 ± 0.16 [4] | 0.785 ± 0.085 [10] | (5.23 ± 0.31) × 10⁷ [10] | 22.1 ± 1.2 [10] |
| EfaB874-888(−) wsHICS10a^{e} | 0.84 [4] | 1.89 ± 0.11 [4] | 0.813 ± 0.022 [4] | (7.05 ± 0.39) × 10⁶ [4] | 22.8 ± 1.1 [4] |
| EfaB874-888(−) wsHICS10b^{d} | 0.72 [4] | 3.34 ± 0.19 [4] | 0.441 ± 0.009 [12] | (4.65 ± 0.26) × 10⁷ [12] | 17.0 ± 0.9 [12] |
| EfaB874-888(−) wsHICS10b^{e} | 0.78 [4] | 3.02 ± 0.23 [4] | 0.460 ± 0.007 [4] | (7.09 ± 0.16) × 10⁶ [4] | 20.6 ± 0.7 [4] |
| EfaB874-888(−) wsHICS11a^{d} | 1.05 [6] | 4.21 ± 0.31 [6] | 0.0878 ± 0.0035 [8] | (2.10 ± 0.09) × 10⁷ [8] | 5.08 ± 0.18 [8] |
| EfaB874-888(−) wsHICS11a^{e} | 1.35 [6] | 3.12 ± 0.08 [6] | 0.0661 ± 0.0017 [6] | (1.30 ± 0.06) × 10⁷ [6] | 5.14 ± 0.25 [6] |
| EfaB874-888(−) wsHICS20a^{d} | 0.75 [6] | 2.44 ± 0.06 [6] | 0.713 ± 0.025 [12] | (5.09 ± 0.19) × 10⁷ [12] | 13.9 ± 0.4 [12] |
| EfaB874-888(−) wsHICS20a^{e} | 1.12 [4] | 1.43 ± 0.02 [4] | 0.687 ± 0.012 [4] | (5.86 ± 0.24) × 10⁶ [4] | 25.6 ± 0.9 [4] |
| EcoB1932-1947(−) HICS18^{d,f} | 0.70 | 3.99 | 0.291 | 3.34 × 10⁸ | 285 |
| CalA1185-1206(−) | 0.98 [20] | 5.69 ± 0.55 [17] | 0.0343 ± 0.0011 | (9.17 ± 0.58) × 10⁷ [17] | 14.0 ± 0.8 [17] |
| wsHICS88^{d} | | | [17] | | |
| CtrB1452-1465(+) HICS51^{d,f} | 0.75 | 2.11 | 0.924 | 1.72 × 10⁸ | 276 |
| NgoA133-145(+) wsHICS63^{d} | 0.97 [14] | 4.92 ± 0.25 [4] | 0.0283 ± 0.0006 | (7.68 ± 0.19) × 10⁷ [14] | 15.7 ± 0.5 [14] |
| | | | [14] | | |

Legend for Table 3:
^{a}Oligonucleotide sequences are in Table 2. Values are mean ± 1 standard deviation from chemical initiation of 0.5 pmol probe hybridized to 1 pmol complementary target in hybridization reagent. Brackets enclose the number of replicates.
^{b}kₐ and k_{b} are from non-linear best fits to Eq. 1 from at least 5 × first order half lives (τ_{1/2}) of each constant.
^{c}Specific activities (SA) are from cps summed over 5 × τ_{1/2} of the decay process divided by the picomoles of probe while signal-to-background ratios (S/B) are from cps summed over 5 × τ_{1/2} of the decay process for probe-target hybrids divided by the same sum for probes alone.
^{d}From light emission detected by PMT without being filtered.
^{e}From light emission detected by PMT after being filtered to >550 nm, specific for emission from ET acceptor.
^{f}Previously reported (Browne, supra).

Rates of increase in cps up to maximum intensity were rapid with τ_{1/2,a}<500 ms, and they varied less than about 3-fold among all probes. Rates of subsequent intensity decrease were much slower and more variable with τ_{1/2,b} ranging up to nearly 25 sec and varying more than 30-fold among all probes. Rate constants for intensity decreases were similar for related chemiluminophores or chemiluminophore-fluorophore conjugates regardless of the nucleic acid backbones comprising the probes, varying up to about 3-fold among the three HICS probes (EfaB874-888(−) HICS1, EcoB1932-1947(−) HICS18 and CtrB1452-1465(+) HICS51), the three wsHICS AE-fluorescein probes (EfaB874-888(−) wsHICS10a, EfaB874-888(−) wsHICS10b and EfaB874-888(−) wsHICS20a) or the three wsHICS AE-tetramethylrhodamine probes (EfaB874-888(−) wsHICS11a, CalA1185-1206(−) wsHICS88 and NgoA133-145(+) wsHICS63). In this sense, k_{b} were better descriptors of the different probe and probe labels than kₐ. When tested (EfaB probe series), total emission and wavelength-resolved emission intensities yielded formation or decay constants similar to within about 2-fold, supporting that the observed total luminescence was primarily due to the wavelength specific emissions. The position of the fluorophore impacted kₐ or k_{b} by a factor of <2-fold for identical sequences in which fluorescein is 5’ of AE (EfaB874-888(−) wsHICS10a), AE is 5′ of fluorescein (EfaB874-888(−) wsHICS10b) or fluorescein is 5′ and one nucleotide removed from AE (EfaB874-888(−) wsHICS20a), indicating tolerance of fluorophore acceptor location.

Specific activities (SAs) were similar (e.g., about 2-5×10⁷ Σcps/pmol) for EfaB874-888(−) HICS1-EfaB874-888(−) wsHICS20a despite a nearly 10-fold variation in the emission decrease rate constants of chemiluminescent emissions (0.088-0.81 sec⁻¹). SAs decreased with increasing shift of emissions to wavelengths higher than those of the fluorescein-containing probes due, at least in part, to decreasing PMT efficiency at higher wavelengths (approaching zero above 650 nm in the current instrument); this efficiency decrease was sufficient to support only minimal detection of emissions from a Cy5 fluorophore (e.g., EfaB874-888(−) wsHICS12a, S/B approaching 1, data not shown). However, the SA for the AE-fluorescein conjugate probes (EfaB874-888(−) wsHICS10a, EfaB874-888(−) wsHICS10b and EfaB874-888(−) wsHICS20a) were slightly higher than for the AE probe (EfaB874-888(−) HICS1) in the same series, suggesting that AE energy transfer to fluorescein may advantageously enhance signal output relative to that of AE alone; this boost tentatively assigned to the high quantum yield of fluorescein. Wavelength-resolved SA varied by up to about 7-fold compared to total SA, likely largely due to a combination of ET and PMT filtering efficiencies. The SA and S/B tended to be lower for the all DNA probes than for the 2′-O-methylribonucleic acid or mixed backbone probes, though sequence context was not taken into account in this assessment. The position of the fluorophore impacted SA and S/B <2-fold for identical sequences in which fluorescein is 5′ of AE, AE is 5′ of fluorescein or fluorescein is 5′ and one nucleotide removed from AE. This finding supports continued use of the synthetically straightforward 3′-quencher-oligonucleotide-AE-fluorophore-5′ probe design. All configurations of AE to fluorophore tested thus far maintain distances between these two moieties substantially closer than the optimal 7-9 nt previously cited for fluorophore-to-fluorophore energy transfer (Hung et al., Anal. Biochem. 1997, 252:78-88).

### 3(c) Wavelength-Resolved Chemiluminescence

Chemiluminescent emission time courses from 200 fmol/100 μL of HICS and wsHICS probes in hybridization reagent were acquired simultaneously on a previously described luminometer modified for dual wavelength-range detection (Browne, supra). Briefly, this luminometer was equipped with two high-count PMT modules (28 mm diameter, head-on, bi-alkali cathode, cathode sensitivity range 300-650 nm, peak cathode radiant sensitivity at 420 nm; Hamamatsu Photonics, Hamamatsu City, Japan) on opposite sides of and directed towards a light-tight detection chamber fitted with injector tubing from two reagent pumps. Changeable filters (25.4 mm dia.; in the present examples, 417/60 BrightLine Bandpass filter (˜95% transmission of light from 385-450 nm) from Semrock, Inc. (Rochester, N.Y.) for HICS probes, and OG550 cut-on filter (˜90% transmission of light from 550-700 nm) from Newport Corporation (Irvine, Calif.) for wsHICS probes) were fitted between the detection chamber and PMT 1 and PMT 2, respectively. Custom Visual Basic software controlled reagent injections and chemiluminescent data acquisition from the two PMT channels. Hybridizations were performed substantially as described in section 3(b), except 50 μL of 0 or 200 amol-200 fmol/50 μL targets in seawater (0.2 μm filtered; Scripps Institution of Oceanography pier, La Jolla, Calif.) were mixed with 50 μL of 2× hybridization reagent containing 200 fmol/50 μL of both probes. Chemiluminescence was initiated by a 200 μL injection of Detect 1 solution followed by a 2 sec pause and then a 200 μL injection of Detect 2 solution Data collection started immediately and continued for 230×0.8 sec intervals (184 sec total) without inter-interval delays. Final pH after detection was always 9.0.

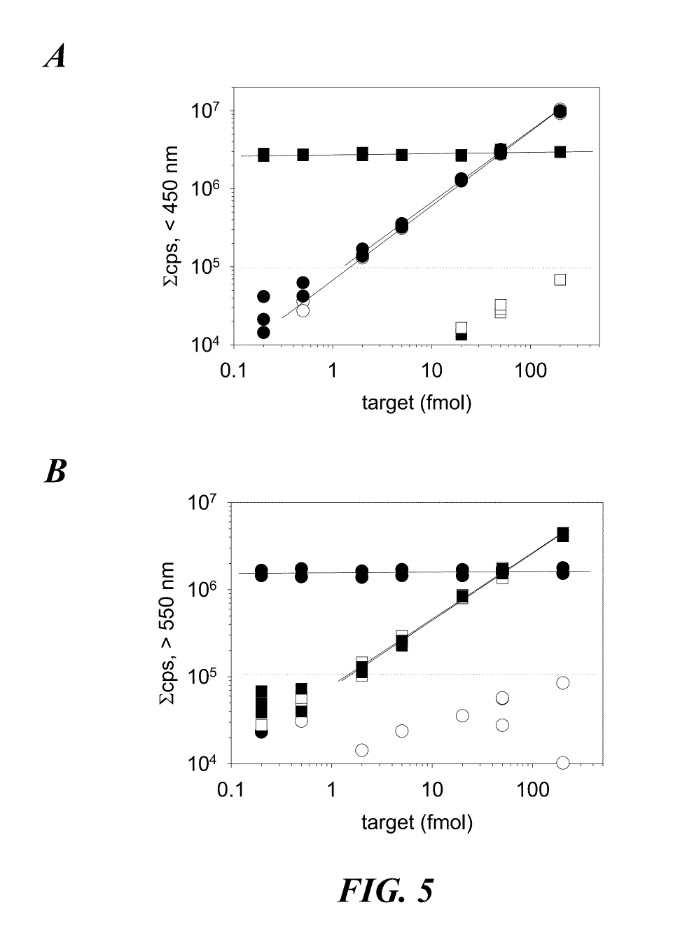

Chemiluminescence measured with a dual wavelength luminometer demonstrated simultaneous, direct detection of bacterial and fungal ribosomal RNA (rRNA) sequences in seawater as simulated indicators of bacteria and fungi quantities in a near shore oceanic environment (FIG. 5). The tetramethylrhodamine label for the pan-fungal probe was selected from wsHICS probe characteristics (based on work with the EfaB probe series) that were spectrally-distinguishable from the pan-bacterial HICS probe while yielding substantial S/B (FIG. 3, Table 3). There was some spectral overlap from high concentrations of the wsHICS probe—target hybrid in the low wavelength channel, and vice versa.

To mitigate this overlap, differences in decay kinetics of the two probes were advantageously weighted by summing early time points from the rapidly emitting HICS probe signal in the low wavelength channel while summing the remaining later time points in the high wavelength channel for the slower emitting wsHICS probe, resulting in spectral-temporal resolution of the signals. Chemiluminescence from a mixture of EcoB1932-1947(−) HICS18 and CalA1185-1206(−) wsHICS88 in solutions containing equal volumes of hybridization reagent and seawater increased after hybridization to increasing concentrations of Eco or Cal rRNA sequences. In the absence or presence of the Cal target, curves for increasing Eco target in the low wavelength channel increased linearly from about 2-200 fmol, with the lower end of the range limited by signals from bleed-through of very high concentrations of Cal target (FIG. 5A). Maintaining the Eco target constant at 50 fmol and increasing Cal target from 0.2-200 fmol yielded a line of zero slope, essentially the same as if no Cal target had been added. Cal target yielded similar quantitative dynamic range and limit of detection results in the high wavelength channel (FIG. 5B). Thus, sequences from either or both pan-generic targets could be simultaneously quantified from about 2-200 fmol in a 50 μL sample within about 60 min.

While not wishing to be bound by any particular mechanism, the predictable changes of emission wavelengths, and substantial emission kinetics and quenching changes, from wsHICS probes relative to HICS probes (FIG. 3, Table 3) provide some insight into the mechanism of chemically-initiated energy transfer to fluorophores. If ET and/or excited fluorophore decay is fast compared to N-alkylacridone* decay (and at 10⁹ sec⁻¹, the latter is certainly true), k_{b} will be dependent on N-alkylacridone* decay rate and will therefore be similar for all probes. Using ET to fluorescein (EfaB874-888(−) wsHICS10a, EfaB874-888(−) wsHICS10b and EfaB874-888(−) wsHICS20a) as an example motif is consistent with this supposition as k_{b} for these probes differed less than about 50% from that of EfaB874-888(−) wsHICS1. However, k_{b} values from AE-tetramethylrhodamine conjugates (e.g., EfaB874-888(−) wsHICS11a, CalA1185-1206(−) wsHICS88 and NgoA133-145(+) wsHICS63) were much lower (about 6- to 32-fold) than directly from N-alkylacridone* of similar nucleic acid backbone probes (e.g., EfaB874-888(−) HICS1, EcoB1932-1947(−) HICS18 and CtrB1452-1465(+) HICS51). If fluorophore wavelength emission depends on overlap between the N-alkylacridone* emission spectrum and fluorophore excitation spectra (resonance ET), longer wavelength fluorophores should yield substantially decreasing emission intensity. However, as the spectrograms demonstrate, total emission from wsHICS probes was high, even when the overlap integral between the donor emission and the acceptor absorption spectra is near zero (FIG. 3A versus FIG. 3D). In the absence of target, ET to the quenching moieties can be, at the very least, from the N-alkylacridone* (FIG. 3A), but may also be from the fluorophores. If the quenching moieties capture energy from the primary emitter, then quenching should be similar for HICS and wsHICS probes (especially within a series of identical sequences). As evidenced by background time-resolved spectrograms and S/B, probes without fluorophores (e.g., EfaB874-888(−) HICS1, EcoB1932-1947(−) HICS18 and CtrB1452-1465(+) HICS51) quench emissions more efficiently than probes that include tetramethylrhodamine near the N-alkylacridone* (e.g., EfaB874-888(−) wsHICS11a, CalA1185-1206(−) wsHICS88 and NgoA 133-145(+) wsHICS63). Overall, these results support that at least some of the fluorophores are excited from the N-alkylacridone* by a radiationless mechanism other than Förster-type resonance ET and that Dabcyl and BHQ2 quench wsHICS emissions from the fluorophores rather than from the N-alkylacridone*; additional studies are needed to identify the energy transfer mechanism.

In summary, using easily designed and synthesized chemiluminescent probes, 2-200 fmol of bacterial and/or fungal analyte nucleic acid sequences representing environmentally relevant sequences were detected within about 1 hour without the requirement for culture, enzymatic amplification, or separation of bound from non-bound probes. In conjunction with the design principles for spectral-temporal resolved chemiluminescent probes described herein, further advances in luminometer technology (e.g., broader PMT wavelength sensitivity range, high transmittance filter sets, narrow band PMTs, rapid spectral detectors used with emission deconvolution algorithms) should facilitate simultaneous differential chemiluminescence detection of three or more distinct probe-target complexes present at even lower concentrations in the near future.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

1. A chemiluminescent probe for detecting a target nucleic acid sequence, the probe comprising:
a target binding region having a base sequence that is substantially complementary to a portion of the target nucleic acid sequence;
an acridinium ester (AE) moiety attached to a first region flanking the target binding region;
a luminophore coupled to the AE moiety to allow energy transfer from an acridone moiety, produced by a chemical triggering of the AE moiety, to the luminophore; and
a quencher moiety attached to a second region flanking the target binding region, such that the first and second flanking regions are on the opposite sides of the target binding region,
wherein in the presence of the target nucleic acid sequence, the probe is altered from an inactive conformation, wherein the quencher moiety is sufficiently proximal to the AE-coupled luminophore to substantially block emission therefrom, to an active conformation, wherein emission from the luminophore can be detected following the triggering of the AE moiety.

2. The probe of claim 1, wherein the AE moiety is conjugated through an acridinium position other than C9 to a stem-loop oligonucleotide.

3. The probe of claim 2, wherein the AE moiety is conjugated through the N10 acridinium position to a stem-loop oligonucleotide.

4. The probe of claim 3, wherein the AE moiety is a 9-(2,6-dibromophenoxycarbonyl)-10-(3-carbonylpropyl)acridinium salt.

5. The probe of claim 1, wherein the luminophore is a fluorophore.

6. The probe of claim 5, wherein the fluorophore is selected from the group consisting of a fluorescein dye, a rhodamine dye, and a cyanine dye.

7. The probe of claim 1, wherein the luminophore is coupled to the AE moiety directly or via a linker.

8. The probe of claim 1, wherein the quencher moiety comprises Dabcyl or Black Hole Quencher 2 (BHQ-2).

9. The probe of claim 1, wherein the probe has a secondary structure selected from the group consisting of the secondary structure of a molecular beacon, the secondary structure of a molecular torch, and the secondary structure of a hybridization switch probe.

10. The probe of claim 1, wherein the target nucleic acid sequence is selected from the group consisting of an Enterococcus target sequence, a pan-fungal target sequence, and a Neisseria gonorrhoeae (N. gonorrhoeae) target sequence.

11. The probe of claim 10, wherein the Enterococcus target sequence comprises an Enterococcus faecalis (E. faecalis) 23S rRNA fragment consisting of SEQ ID NO:1, allowing for a DNA equivalent thereof.

12. The probe of claim 11, comprising a stem-loop oligonucleotide having a target complementary base sequence consisting of SEQ ID NO:6, allowing for an RNA equivalent thereof.

13. The probe of claim 12, wherein the stem-loop oligonucleotide comprises a base sequence consisting of any one of SEQ ID NOs:11 and 12, allowing for RNA equivalents thereof.

14. The probe of claim 10, wherein the pan-fungal target sequence comprises a Candida albicans (C. albicans) 18S rRNA fragment consisting of SEQ ID NO:3, allowing for a DNA equivalent thereof.

15. The probe of claim 14, comprising a stem-loop oligonucleotide having a target complementary sequence consisting of SEQ ID NO:8, allowing for an RNA equivalent thereof.

16. The probe of claim 15, wherein the stem-loop oligonucleotide comprises a base sequence consisting of SEQ ID NO:14, allowing for an RNA equivalent thereof.

17. The probe of claim 10, wherein the N. gonorrhoeae target sequence comprises a 16S rRNA fragment consisting of SEQ ID NO:5, allowing for a DNA equivalent thereof.

18. The probe of claim 17, comprising a stem-loop oligonucleotide having a target complementary base sequence consisting of SEQ ID NO:10, allowing for a DNA equivalent thereof.

19. The probe of claim 18, wherein the stem-loop oligonucleotide comprises a base sequence consisting of SEQ ID NO:16, allowing for a DNA equivalent thereof.

20. A kit for detecting and/or quantifying a target nucleic acid sequence in a sample, the kit comprising the probe of claim 1 and reagent means for triggering a chemiluminescence reaction.

21. A kit for detecting and/or quantifying a plurality of target nucleic acid sequences in a sample, the kit comprising a plurality of probes according to claim 1 and reagent means triggering a chemiluminescence reaction.

22. The kit of claim 21, wherein each of the plurality of probes comprises a different luminophore coupled to the same AE moiety.

23. The kit of claim 22, wherein each of the luminophores is a fluorophore.

24. The kit of claim 23, wherein the fluorophore is selected from the group consisting of a fluorescein dye, a rhodamine dye, and a cyanine dye.

25. The kit of claim 22, wherein the luminophores have sufficiently different emission profiles to allow spectral and/or temporal resolution of the luminescence emissions.
